(19) United States
(12) Patent Application Publication
(10) Pub. No.: US 2024/0175071 A1
(43) Pub. Date: May 30, 2024

(54) METHODS FOR ASSESSING CELLULAR OR TISSUE INJURY USING MITOCHONDRIAL AND/OR NUCLEAR CELL-FREE DNA

(71) Applicant: The Medical College of Wisconsin Inc., Milwaukee, WI (US)

(72) Inventors: Michael Mitchell, Elm Grove, WI (US); Aoy Tomita Mitchell, Elm Grove, WI (US)

(21) Appl. No.: 18/497,541

(22) Filed: Oct. 30, 2023

Related U.S. Application Data
(63) Continuation of application No. PCT/US2022/026799, filed on Apr. 28, 2022.
(60) Provisional application No. 63/181,941, filed on Apr. 29, 2021.

(51) Int. Cl. C12Q 1/68

(52) U.S. Cl. CPC C12Q 1/68; G01N 2800/50; G01N 2800/52

(57) ABSTRACT

This invention relates to methods and compositions for assessing or monitoring an amount of mitochondrial cell-free DNA and/or nuclear cell-free DNA from a subject to assess cellular or tissue injury and/or risk in the subject. The amount(s) can be compared to specific threshold values, in some embodiments, to assess the subject and/or guide treatment of the subject.

### RELATED APPLICATION

This application claims the benefit of priority under 35 U.S.C. § 119 to U.S. Provisional Application No. 63/181,941, filed Apr. 29, 2021 the entire contents of which are incorporated herein by reference.

### FIELD OF THE INVENTION

This invention relates to methods and compositions for assessing an amount of mitochondrial cell-free nucleic acids and optionally, nuclear cell-free DNA, in samples from a subject in which there is cellular or tissue injury. Such amounts can be used to assess and/or monitor the subject to assess the condition and/or risk in the subject.

### SUMMARY OF INVENTION

The present disclosure is based, at least in part, on the surprising discovery that severity and/or risk, such as of a condition and/or complication, associated with cellular or tissue injury, such as surgery (e.g., cardiac surgery) can be correlated with the amount of mitochondrial cell-free DNA (mcf-DNA) and optionally, in combination with nuclear cell-free DNA (ncf-DNA). It has also been found that certain threshold values can be particularly meaningful for assessing risk. It has further been found that there is optimal timing to assess risk. Thus, monitoring amounts of these nucleic acids and comparing to these threshold values can be beneficial to assess a subject and allow for any needed intervention. Provided herein are methods, compositions and kits related to such a determination. The methods, compositions, or kits can be any one of the methods, compositions, or kits, respectively, provided herein, including any one of those of the Examples or Figures.

In one embodiment of any one of the methods provided, the method further comprises obtaining a sample from the subject.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

In one aspect, a report or database comprising one or more of the amounts provided herein is provided.

In one aspect, a method of treating a subject, determining a treatment regimen for a subject or providing information about a treatment to the subject, based on the amount of mitochondrial cell-free DNA and/or nuclear cf-DNA or any one of the methods of analysis provided herein is provided. In one embodiment of any one of such methods, the method comprises a step of treating the subject or providing information about a treatment to the subject. In one embodiment of any one of the methods of treating, the treatment may be any one of the treatments provided herein. In one embodiment of any one of the methods of treating, the treatment is for any one of the conditions provided herein. Examples of which are provided herein or otherwise known to those of ordinary skill in the art.

In one embodiment of any one of the methods provided herein, the method consists essentially of or consists of measurements of mcf-DNA without measurements of ncf-DNA.

In one embodiment of any one of the methods provided herein, the method consists essentially of or consists of measurements of mcf-DNA and ncf-DNA.

In one embodiment of any one of the methods provided herein, any one of the methods provided herein may be a method of treating a subject, such as a cardiac surgical subject (e.g., pediatric or adult).

In one embodiment of any one of the methods provided herein, the threshold to compare the mcf-DNA and/or ncf-DNA may be one of the thresholds provided herein, respectively.

In one embodiment of any one of the methods provided herein, the determination and/or comparison of an amount of mcf-DNA and/or ncf-DNA may be done 12 hours or more after the cellular or tissue injury, such as after surgery. In one embodiment of any one of the methods provided herein, the determination and/or comparison of an amount of mcf-DNA and/or ncf-DNA may be done 12 hours or more and/or 24 hours or more, respectively, after the cellular or tissue injury, such as after surgery.

### BRIEF DESCRIPTION OF FIGURES

The accompanying figures are not intended to be drawn to scale. The figures are illustrative only and are not required for enablement of the disclosure.

FIG. 1 is two time course graphs demonstrating nuclear cell-free DNA (ncf-DNA) kinetics in survivors (lower graph) and those who died (upper graph).

FIG. 2 is a boxplot comparing ncf-DNA levels at 12 hours after cardiopulmonary bypass (CPB) in patients who survived and died.

FIG. 3 shows cutpoint determination for ncf-DNA samples from patients 12 hours after CPB.

FIG. 4 shows boxplots comparing ncf-DNA levels at 12 hours that are less than or equal to 50 ng/mL or greater than 50 ng/mL and, from left to right, the duration of CPB, aortic cross clamp (Aox), and maximum vasoactive inotropic score (VIS).

FIG. 5 is two time course graphs demonstrating mitochondrial cell-free DNA (mcf-DNA) kinetics in survivors (lower graph) and those who died (upper graph).

FIG. 6 is a boxplot comparing mcf-DNA levels at 12 hours after cardiopulmonary bypass (CPB) in patients who survived and died.

FIG. 7 shows cutpoint determination for mcf-DNA samples from patients 12 hours after CPB.

FIG. 8 shows boxplots comparing ncf-DNA levels at 12 hours that are less than or equal to 17 copies/μL or greater than 17 copies/μL and, from left to right, the duration of CPB, aortic cross clamp (Aox), and maximum vasoactive inotropic score (VIS).

FIG. 9 shows a bivariate analysis (ncf-DNA and mcf-DNA).

FIGS. 10A-10D are graphs showing ncf-DNA levels (FIGS. 10A and 10C) and mcf-DNA levels (FIGS. 10B and 10D) in subjects with and without CAED over time.

FIGS. 11A-11B show cutpoint determinations (ROC analyses) for patients less than a year of age (FIG. 11A) or all ages less than 10 years of age (FIG. 11B).

FIG. 12 is a scatterplot of ncf-DNA and mcf-DNA samples collected 12 hour post-CPB according to death (squares) and prolonged length of stay (LOS) in the hospital (circles). The diamonds represent patients who are alive with no prolonged LOS.

### DETAILED DESCRIPTION OF THE INVENTION

It has been found that mitochondrial cell-free DNA and optionally, nuclear cf-DNA is correlated with cellular or tissue injury and can be used to assess and/or monitor a subject in a number of instances, such as in the surgical context. Measuring mitochondrial cf-DNA and/or nuclear cf-DNA can rapidly and effectively assist the clinician in making assessments and can save lives. In addition, monitoring mitochondrial cf-DNA and/or nuclear cf-DNA can be a helpful measure of the effectiveness of therapy.

Mitochondrial cf-DNA and/or nuclear cf-DNA can be a marker for cellular or tissue injury and/or inflammation, respectively, and may be used to guide patient care decisions. In one embodiment, any one of the methods of treatment provided herein can be of a subject identified with the methods of assessment as provided herein. In another embodiment, any one of the methods of treatment provided herein is of a subject determined to have a mitochondrial cf-DNA and/or nuclear cf-DNA amount as provided herein.

As used herein, “mitochondrial cell-free DNA” (or “mitochondrial cf-DNA”) is the amount of mitochondrial cf-DNA present in a sample. Provided herein are methods and compositions that can be used to measure mitochondrial cf-DNA which may then be used to assess the subject's risk associated with cellular or tissue injury. “Nuclear cell-free DNA” (or “nuclear cf-DNA”) is the amount of nuclear cf-DNA present in a sample.

Any one of the methods or compositions provided herein may be used on a sample from a subject that has undergone a surgery, such as cardiac surgery. Such a subject may be a pediatric or adult subject.

Amounts of mitochondrial cf-DNA and/or nuclear cf-DNA, can be used to assess or determine risk or prognosis of a subject having or suspected of having cellular or tissue injury and/or inflammation, such as a surgical subject. As used herein, “suspected of having” refers to a subject whereby a clinician believes there is a likelihood the subject has a specific condition or state. The methods provided herein can be used to confirm a finding or monitor a subject for worsening or improving condition.

In some embodiments, any one of the methods can be used to assess a subject that has had or is at risk of having cellular or tissue injury, a surgical complication and/or inflammation. In some embodiments, the methods provided herein can be used to confirm a finding or monitor a subject for worsening or improving condition.

An amount of mitochondrial cf-DNA and/or nuclear cf-DNA may be determined with experimental techniques, such as those provided elsewhere herein. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e., by purchasing), in some embodiments.

Amounts of mitochondrial cf-DNA and/or nuclear cf-DNA can be used to assess subjects. Thus, a risk of improving or worsening condition can be determined in such subjects. A “risk” as provided herein, refers to the presence or absence or progression of any undesirable condition in a subject, or an increased likelihood of the presence or absence or progression of such a condition. In some embodiments, the risk is a risk of a near-term adverse event. Examples of near-term adverse events include: cardiac arrest, the need for mechanical circulatory support (MCS), and/or death. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or non-progression) of such a condition.

As provided herein, early detection or monitoring can facilitate treatment and improve clinical outcomes. Any one of the methods provided can be performed on any one of the subjects provided herein. Such methods can be used to monitor a subject over time, with or without treatment. Further, such methods can aid in the selection, administration and/or monitoring of a treatment or therapy. Accordingly, the methods provided herein can be used to determine a treatment or monitoring regimen.

“Determining a treatment regimen”, as used herein, refers to the determination of a course of action for treatment of the subject. In one embodiment of any one of the methods provided herein, determining a treatment regimen includes determining an appropriate therapy or information regarding an appropriate therapy to provide to a subject. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

Treatment may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary, by parenteral nutrition—can also be included particularly during prolonged illness. Other associated therapies can include insulin and medication to prevent deep vein thrombosis and gastric ulcers.

In another embodiment, the treatment can be a treatment for infection. In some embodiments, therapies for treating infection include therapies for treating a bacterial, fungal and/or viral infection. Such therapies include antibiotics. Other examples include, but are not limited to, amebicides, aminoglycosides, anthelmintics, antifungals, azole antifungals, echinocandins, polyenes, diarylquinolines, hydrazide derivatives, nicotinic acid derivatives, rifamycin derivatives, streptomyces derivatives, antiviral agents, chemokine receptor antagonist, integrase strand transfer inhibitor, neuraminidase inhibitors, NNRTIs, NS5A inhibitors, nucleoside reverse transcriptase inhibitors (NRTIs), protease inhibitors, purine nucleosides, carbapenems, cephalosporins, glycylcyclines, leprostatics, lincomycin derivatives, macrolide derivatives, ketolides, macrolides, oxazolidinone antibiotics, penicillins, beta-lactamase inhibitors, quinolones, sulfonamides, and tetracyclines.

Other such therapies are known to those of ordinary skill in the art.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

The treatment and clinical course may be determined based on the subject's condition as determined as provided herein and/or the subject's associated expected outcome. For example, if the amount of mitochondrial cf-DNA is 10 copies/μl or greater, 25 copies/μl or greater, 35 copies/μl or greater, or 50 copies/μl or greater, the subject may be treated with, or provided information related thereto, a therapy, such as those described herein. As another example, if the amount of mitochondrial cf-DNA is 17 c/μl or greater, the subject may be treated with, or provided information related thereto, a therapy, such as those described herein. As still another example, if the amount of nuclear cell-free DNA is or 10 ng/mL or greater, 25 ng/mL or greater, or 50 ng/mL or greater, the subject may be treated with, or provided information related thereto, a therapy, such as those described herein. As a further example, if the amount of mitochondrial cf-DNA is 10 copies/μl or greater, 25 copies/μl or greater, 35 copies/μl or greater, or 50 copies/μl or greater (or 17 c/μl or greater), and the amount of nuclear cell-free DNA is or 10 ng/mL or greater, 25 ng/mL or greater, or 50 ng/mL or greater, the subject may be treated with, or provided information related thereto, a therapy, such as those described herein.

“Determining a monitoring regimen”, as used herein, refers to determining a course of action to monitor a condition in the subject over time. In one embodiment of any one of the methods provided herein, determining a monitoring regimen includes determining an appropriate course of action for determining the amount of mitochondrial cf-DNA and/or nuclear cf-DNA in the subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods provided herein determining a monitoring regimen includes determining the timing and/or frequency of obtaining samples from the subject and/or determining or obtaining an amount of mitochondrial cf-DNA and/or nuclear cf-DNA.

In some embodiments, the mitochondrial cf-DNA and/or nuclear cf-DNA may be quantified within 1, 2, 3, 4, 5, 6, 7, 8, 9, 10, 11, 12, 13, 14 or more days after tissue injury (e.g., surgery), as described herein. In some embodiments, the mitochondrial cf-DNA and/or nuclear cf-DNA may be quantified within 1, 2, 3, 4, 5, 6, 7, 8, 9, 10, 11, 12, 13, 14, 15, 16, 17, 181, 19, 20, 21, 22, 23, 24 or more hours after cardiopulmonary bypass (CPB).

In some embodiments, amounts of mitochondrial cf-DNA and/or nuclear cf-DNA can be plotted over time. In some embodiments, threshold values for the points in time may also be plotted. A comparison with a subject's mitochondrial cf-DNA and/or nuclear cf-DNA levels to threshold values over a period of time can be used to predict risk.

In order to monitor the subject's mitochondrial cf-DNA levels and/or nuclear cf-DNA, samples may be taken thrice daily, twice daily, daily, every other day, every second day, every third day, every fourth day, every fifth day, every sixth day, weekly, every other week, every second week, monthly, or at more frequent intervals for up to 1 week, 2 weeks, 3 weeks, 1 month, 2 months, 3 months, or longer. A clinician may determine that a subject should undergo more frequent sampling if the subject's mitochondrial cf-DNA is above a threshold, such as one provided herein, and/or nuclear cf-DNA is found to increase between time points. If a subject is found to have a mitochondrial cf-DNA amount below a threshold and/or nuclear cf-DNA between time points is decreasing, a clinician may determine that less frequent sampling is sufficient. Timing and/or frequency of monitoring may also be determined by a comparison to one or more threshold values for any one of the methods provided herein rather than in comparison to another point in time. Generally, subjects with high and/or increasing amounts of mitochondrial cf-DNA and/or nuclear cf-DNA require closer monitoring and more frequent sampling. In some embodiments of any one of the methods provided herein, each amount and time point may be recorded in a report or in a database.

As increasing levels mitochondrial cf-DNA and/or nuclear cf-DNA have been found to correlate with an increased risk (e.g., risk of near-term events including cardiac arrest, the need for mechanical circulatory support (MCS), and/or death), a clinician may determine that a subject should undergo more frequent sampling if the subject's mitochondrial cf-DNA and/or nuclear cf-DNA are found to increase between time points. If a subject is found to have decreasing levels of mitochondrial cf-DNA and/or nuclear cf-DNA between time points, a clinician may determine that less frequent sampling is sufficient. Accordingly, if a subject does not show such a decrease, the clinician may determine that additional testing and/or treatment may be necessary.

Timing and/or frequency of monitoring may also be determined by a comparison to threshold values. For example, if the amount of mitochondrial cf-DNA and/or nuclear cf-DNA is equal to or greater any one of the thresholds provided herein and/or is increasing, more frequent sampling may be needed, whereas, if the amount of mitochondrial cf-DNA and/or nuclear cf-DNA is less than any one of the thresholds provided herein, and/or is not increasing, less frequent sampling may be required.

In some embodiments of any one of the methods provided herein, each amount and time point may be recorded in a report or in a database. Threshold values may also be recorded in a report or in a database.

Reports with any one or more of the values as provided herein are also provided in an aspect. Reports may be in oral, written (or hard copy) or electronic form, such as in a form that can be visualized or displayed. Preferably, the report provides the amount of mitochondrial cf-DNA and/or nuclear cf-DNA in a sample. In some embodiments, the report provides amounts of mitochondrial cf-DNA and/or nuclear cf-DNA in samples from a subject over time, and can further include corresponding threshold values in some embodiments.

In some embodiments, the amounts and/or threshold values are in or entered into a database. In one aspect, a database with such amounts and/or values is provided. From the amount(s), a clinician may assess the need for a treatment or monitoring of a subject. Accordingly, in any one of the methods provided herein, the method can include assessing the amount of nucleic acids in the subject at more than one point in time. Such assessing can be performed with any one of the methods or compositions provided herein.

As used herein, “amount” refers to any quantitative value for the measurement of mitochondrial cf-DNA and/or nuclear cf-DNA and can be given in an absolute or relative amount. Further, the amount can be a total amount, frequency, ratio, percentage, etc. As used herein, the term “level” can be used instead of “amount” but is intended to refer to the same types of values.

In some embodiments, any one of the methods provided herein can comprise comparing an amount of mitochondrial cf-DNA and/or nuclear cf-DNA to a threshold value, respectively, to identify a subject at increased or decreased risk. In some embodiments of any one of the methods provided herein, a subject having an increased amount of mitochondrial cf-DNA and/or nuclear cf-DNA compared to a threshold value, respectively, is identified as being at increased risk. In some embodiments of any one of the methods provided herein, a subject having a decreased or similar amount of mitochondrial cf-DNA and/or nuclear cf-DNA compared to a threshold value, respectively, is identified as being at decreased or not increased risk. The threshold may be any one of the thresholds provided herein.

“Threshold” or “threshold value” or “cutpoint”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. The threshold value of any one of the methods provided herein, can be any one of the threshold values provided herein, such as in the Examples or Figures.

In some embodiments, if the amount of nuclear cf-DNA measured is equal to or greater than 8, 9, 10, 11, 12, 13, 14, 15, 16, 17, 18, 19, 20, 25, 30, 35, 40, 45, 50, 55, 60, 65, 70, 75 or 80 ng/mL, then the subject may be determined to be at increased risk, such as of an adverse event. In some embodiments, if the amount of nuclear cf-DNA measured is equal to or greater than 50 ng/mL, then the subject may be determined to be at increased risk, such as of a near-term adverse event (e.g., cardiac arrest, the need for mechanical circulatory support (MCS), and/or death).

In some embodiments, the mitochondrial cf-DNA and/or nuclear cf-DNA threshold value of any one of the methods, reports, databases, etc. provided herein, can be any one of the threshold values provided herein, respectively.

The threshold values can be used for comparisons to make treatment and/or monitoring decisions. For example, if the amount of mitochondrial cf-DNA and/or nuclear cf-DNA is equal to or greater than any one of the threshold values provided herein, respectively, and/or increasing over time, further monitoring or treatment may be indicated.

Any one of the methods provided herein may further include an additional test(s) for assessing the subject, or a step of suggesting such further testing to the subject (or providing information about such further testing). The additional test(s) may be any one of the methods provided herein. The additional test(s) may be any one of the other methods provided herein or otherwise known in the art as appropriate.

The amount of mitochondrial cf-DNA and/or nuclear cf-DNA may be determined by a number of methods. In some embodiments such a method is a sequencing-based method. Mitochondrial cf-DNA and/or nuclear cf-DNA may be analyzed using any suitable next generation or high-throughput sequencing technique.

In some embodiments of any one of the methods provided herein, the method is an amplification-based quantitative assay, such as whereby nucleic acids are amplified and the amounts of the nucleic acids can be determined. Such assays include those whereby nucleic acids are amplified with the primers as described herein, or otherwise known in the art, and quantified. Such assays include simple amplification and detection, hybridization techniques, separation technologies, such as electrophoresis, next generation sequencing and the like.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau. In some embodiments, the quantitative PCR is designed to target a region of the mitochondrial genome, for example, the D-loop region.

As used herein, the sample from a subject can be a biological sample. Examples of such biological samples include whole blood, plasma, serum, etc. In some embodiments, addition of further nucleic acids, e.g., a standard, to the sample can be performed.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—Nuclear and Mitochondrial Cell-Free DNA (Independently and in Combination) Predict Death after Infant Cardiac Surgery

Mortality after pediatric cardiopulmonary bypass (CPB) has declined but plateaued. Global inflammation (SIRS) and myocardial dysfunction (e.g., low cardiac output syndrome, LCOS) remain sources of major morbidity and mortality. Development of advanced surgical and perioperative support strategies during pediatric cardiac surgery with cardio-pulmonary bypass have resulted in improved survival, however both overall and high-risk surgical mortality rates have plateaued. The cause can be attributable to both global inflammation or systemic inflammatory response syndrome and myocardial dysfunction or LCOS. The pathogenesis of post-CPB SIRS and LCOS generally involve pathways that injure endothelial and myocardial cells.

Quantification of cell-free DNA (cfDNA) has emerged as a promising tool for probing cellular injury from manifold causes. Refined techniques can separately quantify cfDNA from nuclear (ncfDNA) and mitochondrial (mcfDNA) sources. Data suggest that ncfDNA may quantify inflammation, while mcfDNA may correlate more with myocardial injury.

Multiple mechanisms contribute to physiologic vulnerability following cardiac surgery with release of ncfDNA and mcfDNA. Extent of surgery (tissue injury) and cardiopulmonary bypass provoke the inflammatory response. Myocardial ischemia related to aortic cross-clamp and direct trauma can result in myocardial dysfunction. NCfDNA (nuclear cf-DNA) can be released as part of the inflammatory response as Damage-Associated Molecular Patterns (DAMPs) and Neutrophil Extracellular Traps (NETs), which function as pro-inflammatory mediators. DAMPs can initiate the innate immune response through activation of pattern recognition receptors including toll-like receptors (TLRs). NETs can participate in the inflammatory response to microorganisms. NCfDNA levels can also correlate with the degree of endothelial cell dysfunction, a key component of vascular tone abnormalities in SIRS. Mitochondria are present in high copy numbers in myocardial cells and levels of circulating mitochondrial cfDNA (mcfDNA) are thought to increase with acute myocardial injury.

Here perioperative ncfDNA and mcfDNA levels in infants undergoing congenital heart surgery with cardiopulmonary bypass (CPB) were measured. It was found that elevated ncfDNA and mcfDNA levels can be associated with mortality, perhaps from different pathways, and combined analysis can have a stronger mortality association.

A prospective observational study of infants weighing >3 kg undergoing planned cardiac surgery with CPB was conducted. Association of mortality to ncfDNA and mcfDNA levels was assessed by logistic regression with cutpoints chosen by ROC curve exploration. Fifty-nine patients were surveyed. The median age of the patients was 122 days (range, 4-354 days) and median weight of the patients was 4.9 kg (range, 3.2-9.1 kg). Five patients required ECMO and there were three deaths (5%).

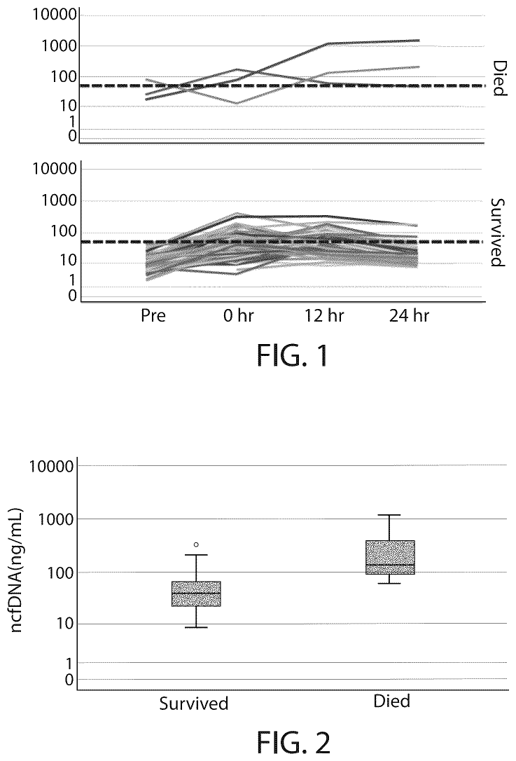

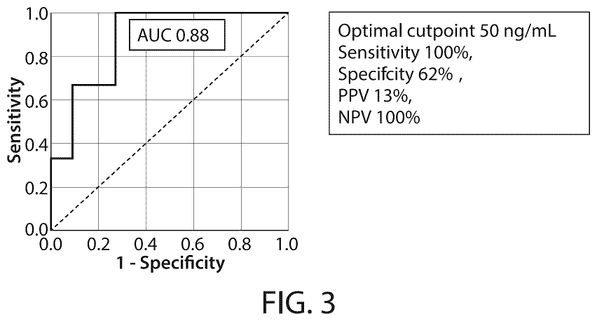

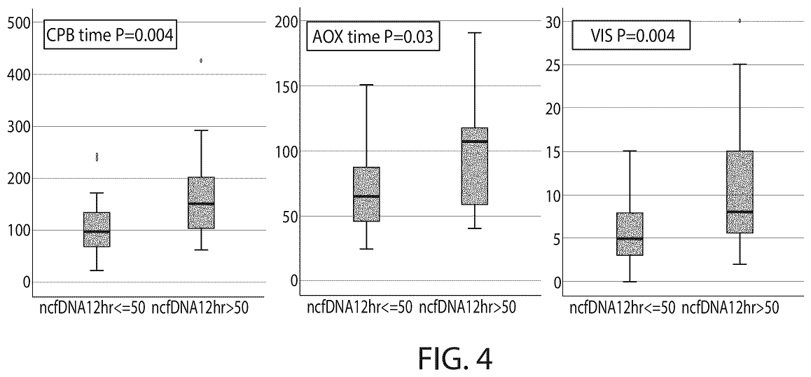

FIG. 1 displays perioperative ncf-DNA kinetics in survivors and those who died. In infants who survived, ncf-DNA levels increased during cardiopulmonary bypass (CPB) and continued to increase though 12 hours before decreasing towards baseline at 24 hours. Those who died exhibited a failure of ncf-DNA to return to baseline. FIG. 2 shows a boxplot comparing ncf-DNA levels at 12 hours after CPB in patients who survived and died and revealed a trend of increased ncf-DNA levels in those that died. Next, receiver operating characteristic (ROC) analysis was performed, comparing ncf-DNA at 12 hours (after CPB) and death. The AUC was 0.88 with and optimal cutpoint of 50 ng/ml. Using this threshold of ncfDNA 12 hours >50 ng/ml, the relationship between ncf-DNA levels and duration of CPB, aortic cross clamp (Aox), and maximum vasoactive inotropic score (max VIS) were examined. It was found that elevated levels of ncf-DNA were associated with longer duration of cardiopulmonary bypass, aortic cross clamp and greater maximum vasoactive inotropic score in the first 24 hours (FIG. 4).

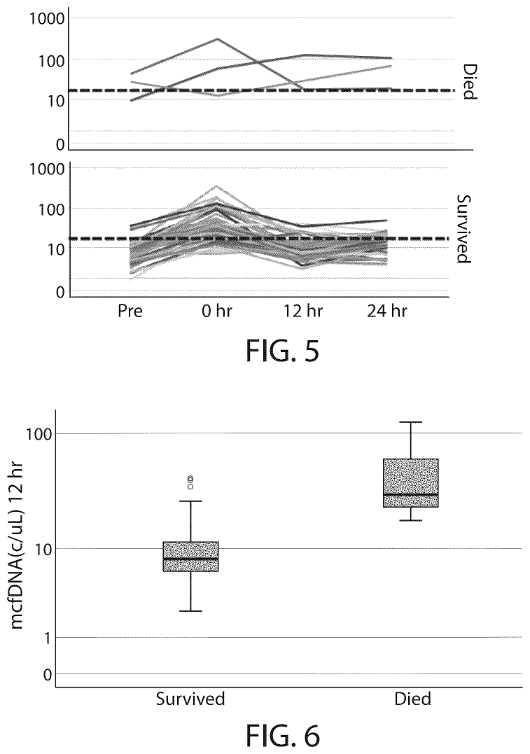

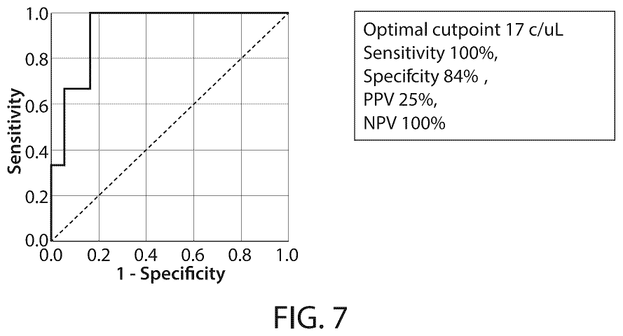

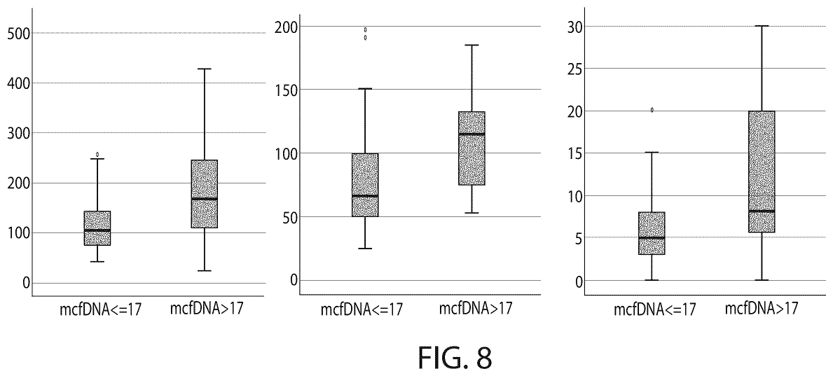

The studies were repeated to examine mitochondrial cf-DNA (mcf-DNA). The kinetics are shown in FIG. 5 and demonstrate that, in contrast to ncf-DNA, mcf-DNA levels in survivors declined much more quickly following CPB, approaching baseline levels within 12 hours. Patients who died exhibited persistent elevation. FIG. 6 is a boxplot demonstrating that 12 hour mcf-DNA levels were significantly higher in those who died than those who survived. The ROC analysis of 12 hour mcf-DNA levels and death generated an AUC of 0.88 with an optimal cutpoint of 17c/μl (FIG. 7). Using this threshold of mcf-DNA 12 hours >17c/μl, the relationship between mcf-DNA levels and the duration of CPB, Aox, and max VIS was examined. It was found that an increased mcf-DNA was associated with longer duration of cardiopulmonary bypass, aortic cross clamp, and a nonsignificant trend of increased maximum 24 hour VIS (FIG. 8).

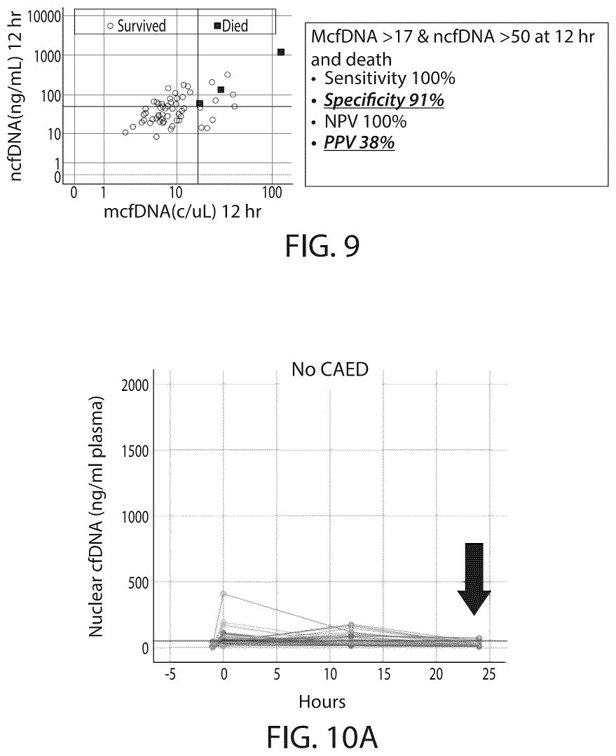

Using the threshold identified by ROC analysis, a bivariate analysis was performed (FIG. 9). It demonstrated improved predictive ability with increased specificity and PPV. The association between elevated early postoperative ncf-DNA and mcf-DNA levels with mortality was assessed by logistic regression with cutpoints chosen by ROC curve exploration. The primary outcome death was met in 3/59 (5%). Ncf-DNA and mcf-DNA levels at 12 hours after the initiation of CPB predicted death at a threshold of 50 ng/ml ncfDNA and 17 copies/ul mcfDNA with 100% sensitivity and 100% negative predictive value (FIG. 12). The specificity (91%) and positive predictive value (38%) increased through combined analysis compared to univariate analysis. Combined analysis exhibited high specificity (93%) and negative predictive value (78%) for prolonged (>30 postoperative days) hospitalization (circles in FIG. 12).

Therefore, analysis of ncf-DNA and mcf-DNA can each be used to stratify risk of mortality following infant cardiac surgery, and risk determination can be improved through bivariate analysis. Evaluation of ncf-DNA and mcf-DNA alone and in combination to identify states of generalized inflammation and myocardial injury can allow for targeted interventions and improved outcomes.

### Example 2—Congenital Heart Surgery Pilot Study

A single center cohort was investigated examining ncf-DNA and mcf-DNA kinetics in pediatric congenital cardiac surgery in patients less than 18 years old, including normative patterns of elevation and decline following separation from CPB. The primary outcome analyzed was the composite outcome of CAED. A total of 703 samples were collected from 117 patients. The median age at the time of surgery was 0.95 (range 0-17.4) years, median weight was 8.1 (range 3.2-97.7) kg.

A qPCR assay was designed and tested targeting the D-loop region of the mitochondrial genome, avoiding known common deletions. Mitochondrial control DNA was generated starting from human induced pluripotent stem cell derived cardiomyocytes that were differentiated. Mitochondria was purified using a mitochondria isolation kit for cultured cells (Thermo Scientific), mitochondrial DNA was extracted using QIAamp DNA minikit (Qiagen) and quantified by digital PCR (BioRad QX100 droplet digital PCR system). Mitochondrial DNA was then used as a mitochondrial DNA standard to determine mitochondrial cfDNA copies/μl plasma.

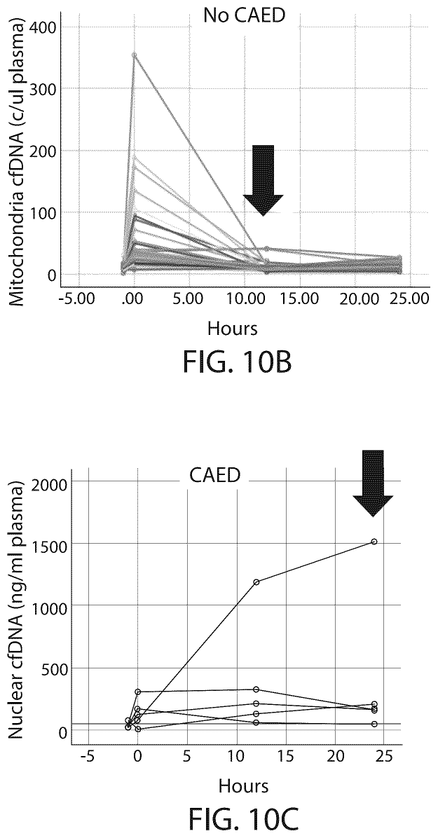

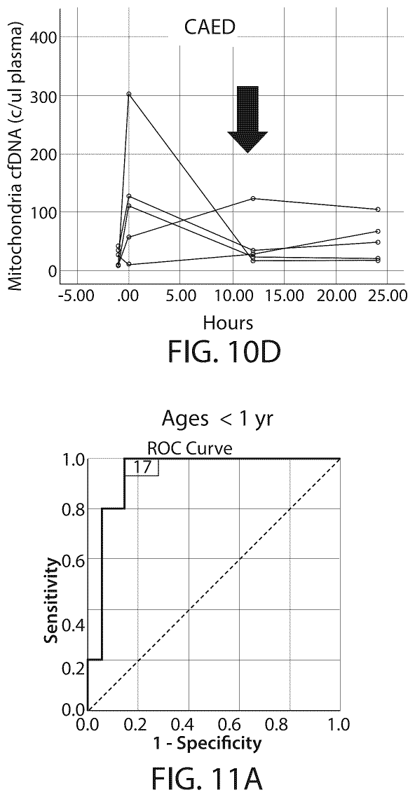

In infants and neonates without CAED, ncf-DNA levels increased during CPB and continued to increase to 12 hours before decreasing towards baseline at 24 hours (FIG. 10A). In contrast, mcf-DNA levels in patients without CAED declined much more quickly following CPB, approaching baseline levels within 12 hours (FIG. 10B). Patients who developed CAED had failure to return to baseline in both ncf.DNA (FIG. 10C) and mcf-DNA (FIG. 10D). The maximally predictive time point was 24 hours for ncf-DNA and 12 hours for mcf-DNA, as indicated by the arrows in FIGS. 10A-10D. Pre-CPB nef-DNA levels were 6.7 (IQR 3.9-11.1) ng/ml and increased immediately following separation from CPB to 44.9 (IQR 20.2-77.2) ng/ml and declined to 14.8 (IQR 10.8-24.9) ng/ml at 24 hours post-operatively.

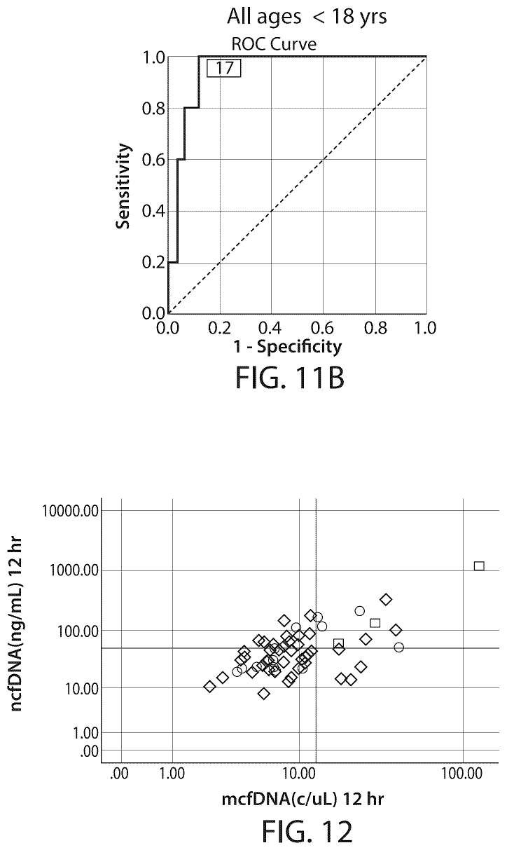

A ROC analysis of mitochondrial cfDNA and CAED is shown in FIGS. 11A-11B. Mcf-DNA at the maximally predictive 12 hour time point at a threshold of 17 c/μl has sensitivity of 100% and specificity >85% for age <1 year (FIG. 11A) and also for all ages <18 years (FIG. 11B).

### Example 3—Nuclear Cell-Free DNA as Predictor Near-Term Adverse Events

A multi-center prospective study was undertaken to investigate the value of nuclear cell-free DNA (ncf-DNA) in non-invasive monitoring following heart transplantation from 388 heart transplant patients. The data demonstrate a correlation between elevated ncf-DNA and near-term events including cardiac arrest, the need for mechanical circulatory support (MCS), and death following adult and pediatric heart transplantation. As such, ncf-DNA has the potential to provide additional information for clinical surveillance, including infection. A pilot prospective observational study of 116 children undergoing cardiac surgery with cardiopulmonary bypass (CPB) pre-operative ncfDNA levels >20 ng/mL independently predicted cardiac arrest, ECMO, and death (CAED) (OR=18.2, CI 2.2-212, p=0.002) as well as prolonged hospital length of stay (LOS) (p<0.01).

1. A method of assessing a sample from a subject that has or is suspected of having cellular or tissue injury, the method comprising:
(a) determining an amount of mitochondrial cell-free DNA (mitochondrial cf-DNA) and/or nuclear cf-DNA in a sample taken from the subject; and
(b) optionally, reporting and/or recording the amount of mitochondrial cf-DNA and/or nuclear cf-DNA.

2. A method of assessing a subject, the method comprising:
(a) obtaining an amount of mitochondrial cell-free DNA (mitochondrial cf-DNA) and/or nuclear cf-DNA; and
(b) comparing the amount of mitochondrial cf-DNA and/or nuclear cf-DNA to a threshold value, respectively, wherein when the amount is greater than or equal to the threshold value(s), risk is indicated; and
(c) optionally, determining a treatment or monitoring regimen for the subject based on the comparison(s).

3. The method of claim 1, wherein one or more further amounts of mitochondrial cf-DNA and/or nuclear cf-DNA are obtained from a sample taken from the subject at a different point in time.

4. A method of assessing a sample from a subject that has undergone, is undergoing, or will undergo cellular or tissue injury or treatment for cellular or tissue injury, the method comprising:
(a) determining an amount of mitochondrial cell-free DNA (mitochondrial cf-DNA) and/or nuclear cf-DNA in at least one sample taken from the subject, wherein the at least one sample is taken prior to cellular or tissue injury and/or taken post cellular or tissue injury; or
(b) determining an amount of mitochondrial cf-DNA and/or nuclear cf-DNA in at least one sample taken from the subject, wherein the at least one sample is taken prior to treatment and/or taken post treatment; and
(c) comparing the amount(s) to a threshold to assess a risk in the subject, wherein when the amount(s) are greater than or equal to the threshold value risk is indicated or increased.

5. (canceled)

6. The method of claim 4, wherein the method further comprises (d) reporting and/or recording the amount(s) of mitochondrial cf-DNA and/or nuclear cf-DNA, optionally wherein the amount(s) are provided in a report or a database.

7. The method of claim 4, wherein at least one sample is taken immediately prior to the cellular or tissue injury or treatment and/or wherein one or more further amounts of mitochondrial cf-DNA and/or nuclear cf-DNA are determined each from a sample taken from the subject at a different point in time, such as a different point in time during the cellular or tissue injury treatment.

8. (canceled)

9. The method of claim 4, wherein the method further comprises:
(d) comparing the amount(s) of mitochondrial cf-DNA and/or nuclear cf-DNA to a threshold value, wherein when the amount(s) are greater than or equal to the threshold value risk is indicated or increased; or
(e) determining a treatment or monitoring regimen for the subject based on the amount(s) of mitochondrial cf-DNA and/or nuclear cf-DNA compared to the threshold value(s); or
(f) comparing the amount(s) of mitochondrial cf-DNA and/or nuclear cf-DNA to a threshold value, wherein when the amount(s) are greater than or equal to the threshold value risk is indicated or increased and determining a treatment or monitoring regimen for the subject based on the amount(s) of mitochondrial cf-DNA and/or nuclear cf-DNA compared to the threshold value(s).

10. (canceled)

11. The method of claim 1, wherein at least one amount of mitochondrial cf-DNA and/or nuclear cf-DNA is determined 12 hours or more after the cellular or tissue injury or wherein at least one amount of mitochondrial cf-DNA is determined 12 or more hours after the cellular or tissue injury and at least one amount of nuclear cf-DNA is determined 24 hours or more after the cellular or tissue injury.

12. (canceled)

13. The method of claim 1, wherein the cellular or tissue injury is surgery, such as cardiac surgery, or cardiopulmonary bypass.

14. (canceled)

15. A report of that comprises the amount(s) of claim 1.

16. (canceled)

17. A database that comprises the amount(s) of claim 1.

18. The method of any claim 9, wherein the determining a monitoring regimen comprises determining the amount of mitochondrial cf-DNA and/or nuclear cf-DNA in the subject over time or at a subsequent point in time, suggesting such monitoring to the subject, or using or suggesting the use of one or more additional test(s) to assess the subject.

19. (canceled)

20. The method of claim 9, wherein the determining a treatment regimen comprises selecting or suggesting a treatment for the subject, changing the treatment of the subject or suggesting such change, treating the subject, or providing information about a treatment to the subject

21. The method of claim 9, wherein the treatment is based on the determination or comparison, optionally wherein the treatment is hemodialysis, mechanical ventilation, transfusion of blood products, or drug and fluid therapy.

22-23. (canceled)

24. The method of claim 1, wherein the subject is a subject that has undergone surgery, such as cardiac surgery, or cardiopulmonary bypass or is one with or suspected to have myocardial injury.

25-26. (canceled)

27. The method of claim 1, wherein the subject is a pediatric subject.

28. (canceled)

29. The method of claim 1, wherein the subject is one that has or is suspected of having a need for mechanical circulatory support (MCS), or is at increased risk of cardiac arrest, needing MCS, death, or any combination thereof.

30. (canceled)

31. The method of claim 4, wherein an amount of mitochondrial cf-DNA and/or nuclear cf-DNA that is greater than a threshold value represents an increased risk, or wherein an amount of mitochondrial cf-DNA and/or nuclear cf-DNA that is lower than the threshold value represents a decreased risk.

32-33. (canceled)

34. The method of claim 2, wherein the threshold of mitochondrial cf-DNA is 10 copies/μl or greater.

35-38. (canceled)

39. The method of claim 2, wherein the threshold nuclear cf-DNA value is 10 ng/ml or greater.

40-41. (canceled)
